(19) United States
(12) Patent Application Publication
(10) Pub. No.: US 2010/0241200 A1
(43) Pub. Date: Sep. 23, 2010

(54) HEATING PAD COVER

(76) Inventors: Mark H. BRUDER, Alpharetta, GA (US); Aaron N. INGRAM, Canton, GA (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 12/727,414

(22) Filed: Mar. 19, 2010

Related U.S. Application Data
(60) Provisional application No. 61/162,439, filed on Mar. 23, 2009.

(51) Int. Cl. A61F 7/08

(52) U.S. Cl. 607/112; 607/114

(57) ABSTRACT

A two-piece heating pad cover formed of a first body and a second body. The first body has an inner layer and an outer layer. The inner layer is secured to the outer layer and forms a heating pad receivable container having a closable opening. The cover has a second body having a heat transfer layer. The first body is removably secured to the second body.

### CROSS-REFERENCE TO RELATED APPLICATION

This application claims the benefit of U.S. Provisional Patent Application Ser. No. 61/162,439, filed Mar. 23, 2009, the entirety of which is hereby incorporated herein by reference for all purposes.

### TECHNICAL FIELD

The present invention relates generally to the field of healthcare and therapeutic devices, and more particularly to covers for thermal treatment products.

### BACKGROUND OF THE INVENTION

Heating pads, ice-packs and other thermal treatment devices are used in a variety of ways to provide hot or cold therapy to human and animal patients, for example to relieve inflammation and pain, to promote circulation and healing, and the like. These products are often used in conjunction with a cover. The cover protects the user from overheating or overcooling that might result from direct contact with the thermal therapy product. The cover also protects the heating pad from contamination by bodily fluids or other substances, and allows for more convenient reuse. The traditional heating pad cover is a single structure that functions similarly to a bag or pillowcase. The traditional heating pad covers are sometimes awkward and difficult to change when use is complete.

### SUMMARY OF THE INVENTION

In one aspect, the present invention is a two piece heating pad cover. It is designed so that one piece remains on the unit between patient use and the second piece, which is optionally made of materials that can wick and store water, is easily removable for rapid changeover between patients. Either piece can be independently laundered. Traditional covers are one piece and are more awkward to change.

In another aspect, the invention is a thermal therapy device. The thermal therapy device has a first component with an outer panel and an inner panel, and forming a chamber therebetween for receiving a thermal therapy pad. The outer panel is insulative, and the inner panel is highly heat transmissive relative to the outer panel. The thermal therapy device also has a second component with an inner face and an outer face. The inner face has at least one detachable coupling for removable attachment of the second component to the first component.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a side elevation view of a two-piece cover according to an example embodiment of the present invention.

FIG. 2 is an interior plan view of the cover shown in FIG. 1, shown with the pieces separated.

FIG. 3 is a top perspective view of an electric heating pad.

FIG. 4 is a top perspective view of a cover according to the example embodiment of FIG. 1 in use with a heating pad with the first body inner layer shown.

FIG. 5 is a bottom perspective view of the cover of FIG. 1 in use with a heating pad with the second body shown.

FIG. 6 is a top perspective view of the cover of FIG. 1 with the first body partially separated from the second body.

FIG. 7 is a top perspective view of the cover of FIG. 6 without the heating pad.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

In an example embodiment, the invention comprises a multi-body or multi-piece cover for a heating pad, ice-pack or other thermal therapy product. The cover will be described herein primarily with regard to use in connection with a heating pad, but will be understood to be adaptable for use with other therapy products for providing heating, cooling, moisture, medication-delivery, antibiotic, and/or other forms of contact therapy to a human or animal subject. Example embodiments include a first body or component, and a second body or component that is releasably attachable to the first body. The first body contains, or secures to, the heating pad, and the second body contacts the subject during treatment, and is easily detached and removed for cleaning or changing for use with a different user.

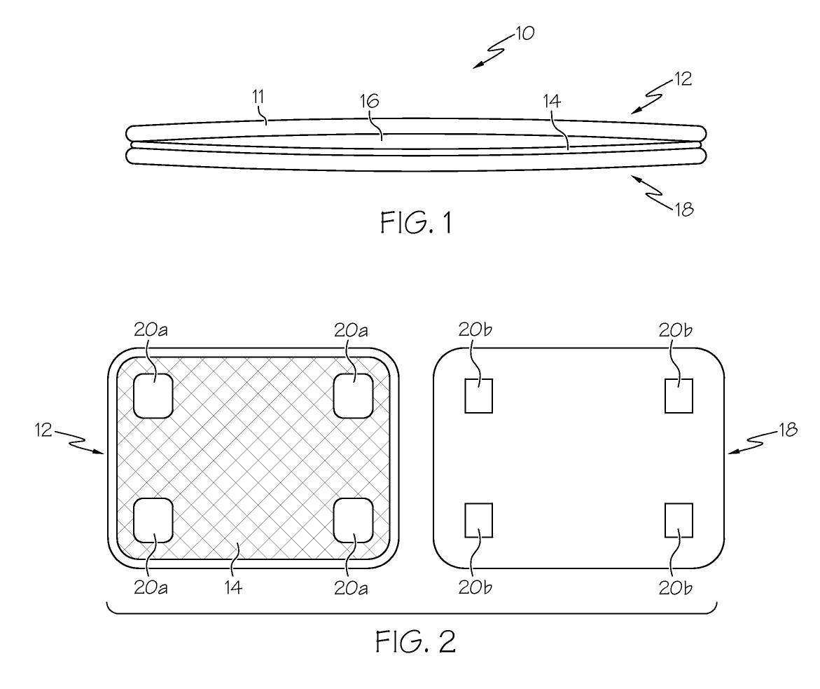

With reference now to the drawing figures, wherein like reference numbers represent corresponding parts throughout the several views, FIG. 1 shows a side view of an example embodiment of a heating pad cover 10 with the first body 12 secured to the second body 18. As shown, the first body 12 has an outer layer 11 and an inner layer 14. When in use, the second body 18 is oriented toward the user and the first body 12 is oriented away from the user. The outer layer 11 faces away from the second body 18 when assembled, and the inner layer 14 faces toward the second body 18.

As shown, a cavity 16 is formed between the inner layer 14 and outer layer 11 of the first body 12. Preferably, the cavity 16 is formed by securing the outer edges of the inner layer 14 to the outer edges of the outer layer 11 along three sides, as for example by stitching, adhesive, integral formation, hook and loop, zipper, buttons, snap fastener or other connection means; and leaving a section along a fourth side unsecured to allow insertion and removal of the heating pad into the cavity 16. The unsecured section is optionally semi-permanently closed by a securing means such as a snap fastener, button, hook and loop fastener system, or other separable connection means. As described below, a heating pad is inserted into the cavity 16 between the first body 12 and second body 18. In alternate forms, the first body comprises an integral portion of a heating pad or other contact therapy item.

The outer layer 11 of the first body 12 includes a panel of fabric or other flexible material having drapability so that it may conform to the body part or other surface on which it is placed. The material for the outer layer 11 preferably has a relatively low heat capacity so as to insulate against the transfer of heat away from a user. The material for the outer layer 11 can alternatively have heat reflective properties on its inner face to reflect heat toward a user. The inner layer 14 of the first body 12 is preferably a material thin enough to allow the free transfer of heat toward a user. A suitable material for the inner layer 14 is for example an open mesh fabric.

The first body 12 is preferably weighted with a dense material for maintaining contact of the therapeutic device against the subject's body during treatment. The first body 12 can be weighted with a panel that is inserted between the inner 14 and outer layers 11. The first body 12 can alternatively be weighted with an integrated layer that is secured within or that forms the first body 12. The weighting panel is optionally a silicone sheet or a particulate or granular material. The weighting panel is preferably highly flexible and drapable to conform to the shape of the contact surface of a user. The weighting panel can provide a low heat capacity, insulation or heat-reflective property.

The second body 18 is preferably fabricated of a material that has a relatively high heat transfer property. The second body 18 is optionally fabricated of a material that has wicking and moisture storage properties so as to collect humidity from the ambient surroundings and deliver moisture to a user during heat therapy. The second body 18 may, for example, be formed of a wettable or high-loft polyester so as to provide moisture therapy. The material of the second body 18 can alternatively be selected to deliver topical medicine or antibiotic, such as through infusion therapy. The first body 12 and the second body 18 can preferably both be washed, either separately or independently.

In use, the second body 18 contacts the treated body surface of a human or animal subject. The first body remains on the heating pad, and is protected from contact with the subject by the second body. As a result, the second body may become contaminated. The second body can be easily detached from the first body, removed for disposal or washing, and replaced with a new or clean second body allowing rapid changeover between patients for reuse of the overall device. In example forms, the second body 18 can be fabricated from a woven or non-woven material, such as for example, polyester, cotton, nylon, polypropylene, and may be breathable or non-breathable. The second body 18 can optionally include such an outer layer for contact with the subject, and an inner layer of a thin, heat-transmissive material such as mesh. This inner layer contacts and attaches to the inner layer 14 of the first body 12. In alternative forms of the invention, the second body 18 can be either disposable or reusable. The second body 18 can be for single patient use, while the first body 12 is reusable. For example, after use, the second body 18 is detached and disposed of, and a new second body is replaced back onto the first body 12 for subsequent use.

FIG. 2 shows the first body 12 separated from the second body 18. The interior faces of the first body 12 and second body 18 are shown. As shown, the first body 12 has a plurality of connectors or securing means 20a. Preferably, the securing means 20a are located on the inner layer 14 of the first body 12. As shown, the second body 18 has a matching number of cooperating securing means 20b. As shown, there are four securing means pairs 20a, 20b on each of the first body 12 and second body 18, however, alternate amounts of securing means are capable. Preferably, the securing means 20a, 20b are hook-and-loop fastener material or snaps, however alternative means are possible. The first body 12 is removably secured to the second body 18 by aligning and connecting the securing means 20a, 20b to one another. The first body 12 is unsecured from the second body 18 by detaching and separating the securing means 20a, 20b.

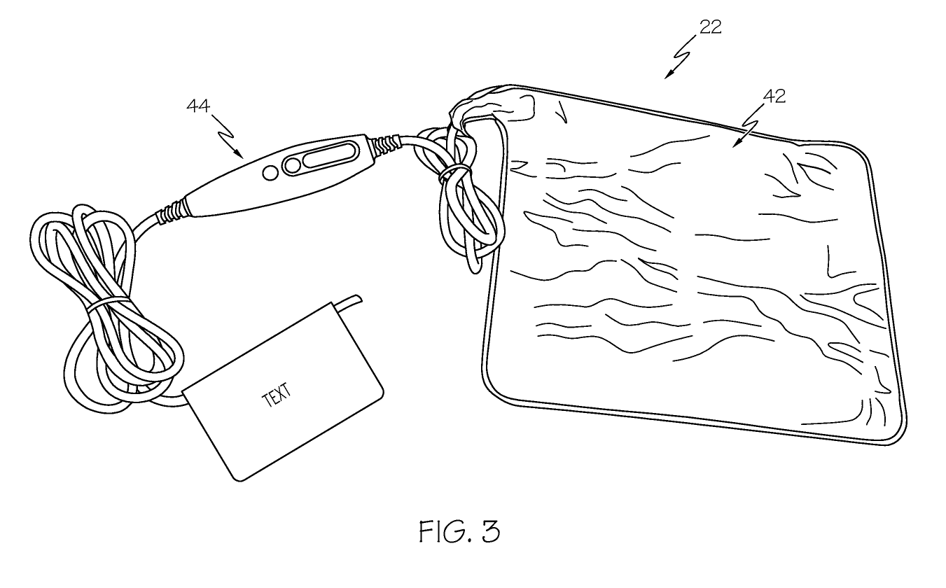

FIG. 3 shows an electric heating pad 22 prior to application of the cover according to the present invention. The heating pad 42 has a flat rectangular heat-producing portion 42 and a power/operation portion 44. The power/operation portion 44 can have a power cord and an operation control. The heating pad 22 is depicted as an electric heating pad, but the present invention is suitable for use in connection with chemical-activated heating or cooling pads, microwave activated heating pads, air activated heating pads, or various other forms of contact therapy products.

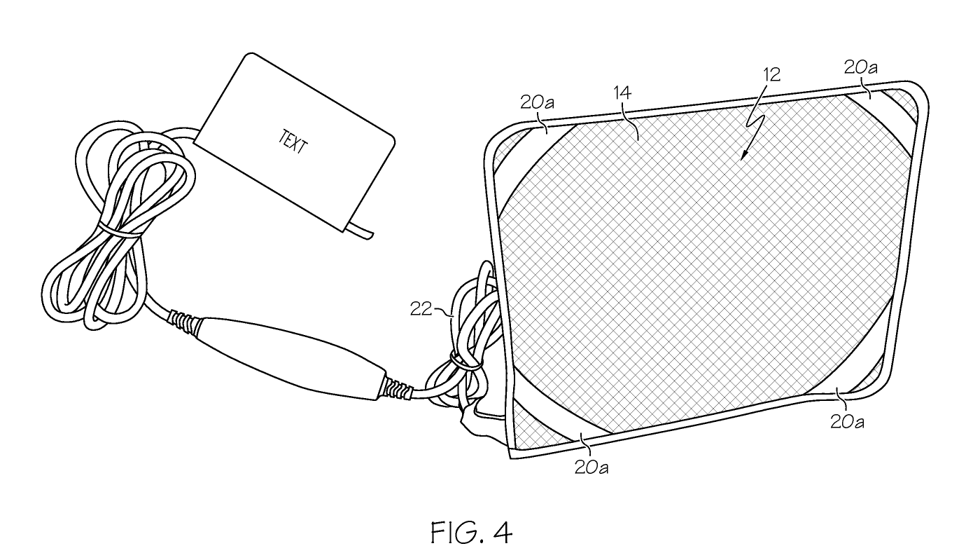

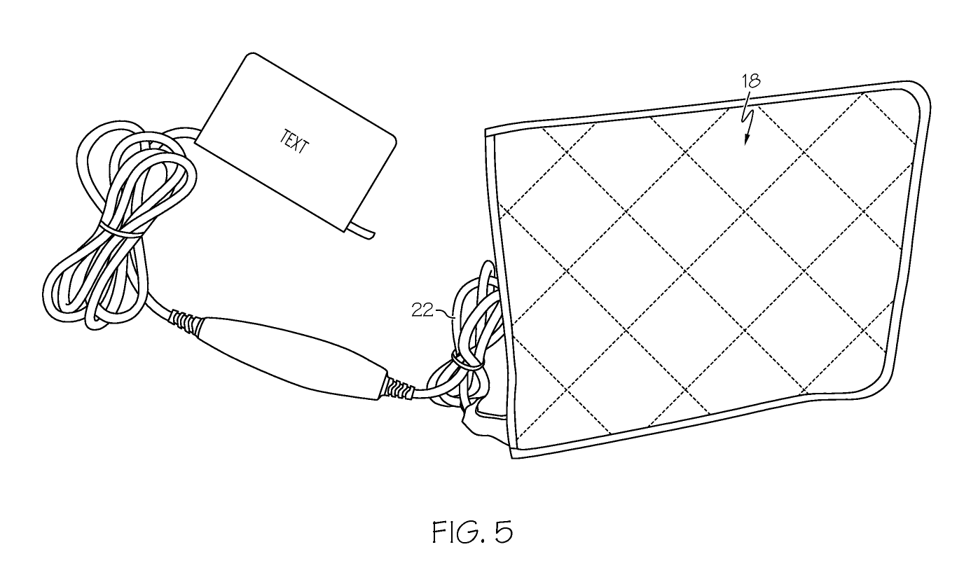

As shown in FIG. 4, the heat-producing portion 42 of the heating pad is inserted into the first body 12 of the cover 10. FIG. 5 shows the heating pad cover 10 in use with a heating pad 22 inserted into the cavity 16 described in FIG. 1. The first 12 and second 18 bodies of the cover 10 preferably have outer peripheries of substantially matching size and shape, for example joining to form a rectangular, circular, or otherwise configured assembly. As shown, the outer surface of the second body 18 can have a padded pattern for added comfort to the user.

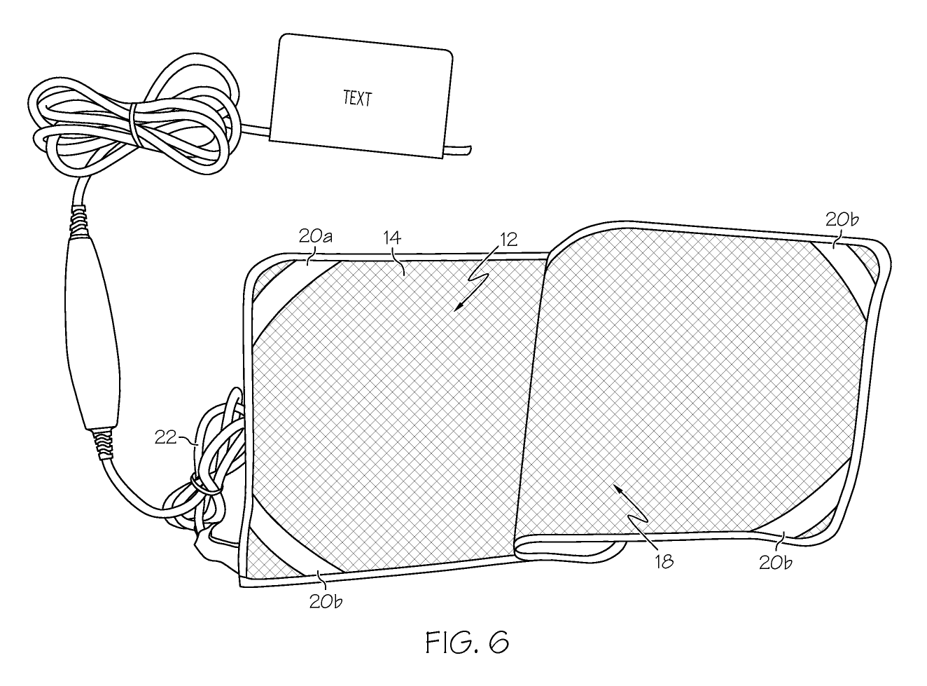

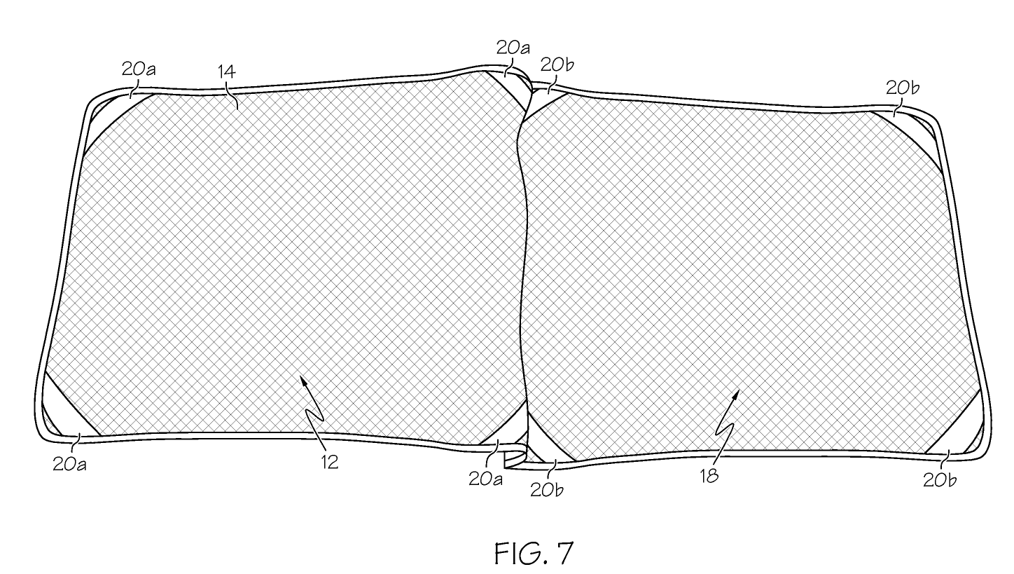

FIG. 6 shows the second body 18 partially detached from the first body 12. As shown, both the first body 12 and the second body 18 can have a mesh interior surface for effective heat transfer. Further, as shown, the heating pad 22 can remain within the cavity of the first body 12 when the heating pad cover 10 is not in use, and, the user can simply separate the second body 18 for cleaning. FIG. 8 shows the first body 12 and the second body 18 secured with two securing means 20a, 20b. As shown, the first body 12 and second body 18 can be secured along one edge and separated from each other to form a larger surface onto which a user can place heating or cooling therapy devices.

While the invention has been described with reference to preferred and example embodiments, it will be understood by those skilled in the art that a variety of modifications, additions and deletions are within the scope of the invention, as defined by the following claims.

What is claimed is:

1. A cover for a contact therapy device, said cover comprising:
a first body comprising an inner layer and an outer layer, wherein the inner layer is secured to the outer layer to form a pocket having an opening for receiving and removing a contact therapy device;
a second body comprising a thermally transmissive contact face;
wherein said second body is removably attachable to said first body.

2. A heating pad cover as in claim 1, wherein said first body is weighted.

3. A heating pad cover as in claim 2, wherein said first body is weighted by a flexible panel of dense material.

4. A heating pad cover as in claim 1, wherein said first body outer layer is thermally insulative or reflective.

5. A heating pad cover as in claim 1, wherein said first body inner layer is heat transmissive.

6. A heating pad cover as in claim 1, wherein the thermally transmissive contact face of said second body is moisture absorbent.

7. A heating pad cover as in claim 1, wherein said first body is removably secured to said second body through securing means.

8. A heating pad cover as in claim 7, wherein said securing means is a hook-and-loop fastener system.

9. A thermal therapy cover comprising:
a first component having an outer panel and an inner panel, and forming a chamber therebetween for receiving a thermal therapy pad, the outer panel being insulative, and the inner panel being highly heat transmissive relative to the outer panel;
a second component comprising an inner face and an outer face, the inner face comprising at least one detachable coupling for removable attachment of the second component to the first component.

10. The thermal therapy cover of claim 9, wherein the inner panel of the first component comprises an open mesh fabric.

11. The thermal therapy cover of claim 9, wherein the inner panel and the outer panel of the first component comprise rectangular panels permanently affixed along three side edges thereof, and having a hook-and-loop closure at the fourth side edge.

12. The thermal therapy cover of claim 9, wherein the second component comprises a heat-transmissive material.

13. The thermal therapy cover of claim 9, wherein the second component comprises moisture-wicking material.

14. The thermal therapy cover of claim 9, wherein the first component comprises a weight incorporation feature.

15. The thermal therapy cover of claim 14, wherein the weight incorporation feature comprises a dense material.

16. The thermal therapy cover of claim 14, wherein the weight incorporation feature comprises a panel removably inserted into the first component chamber.

17. A thermal therapy device comprising:
a flexible first body comprising a heat reflective outer layer secured along a substantially incomplete edge of an inner layer; and
a heat-transferable second body removably secured to the first body inner layer.

18. The thermal therapy device of claim 17, wherein the first body comprises a cavity between the outer layer and the inner layer.

19. The thermal therapy device of claim 18 further comprising a heating device removably inserted into the cavity between the first-body-outer-layer and the first-layer-inner-layer.

20. The thermal therapy device of claim 17, wherein the second body is removably secured to the first-body-inner-layer with a plurality of connectors.

21. A thermal treatment system comprising:
a contact therapy device having a first component attached thereto;
a plurality of second bodies for replaceable attachment to the first component; and
releasable attachment means for detachably securing one of the plurality of second bodies to the first component.

22. The thermal treatment system of claim 21, wherein the contact therapy device is a heating pad.
